(19) United States
(12) Plant Patent Application Publication
(10) Pub. No.: US 2014/0007310 P1
(43) Pub. Date: Jan. 2, 2014

(54) Heuchera plant named 'Galaxy'

(50) ;

(71) Applicant: Janet N. Egger, Wilsonville, OR (US)

(72) Inventor: Janet N. Egger, Wilsonville, OR (US)

(73) Assignee: Terra Nova Nurseries, Inc.

(21) Appl. No.: 13/507,430

(22) Filed: Jun. 27, 2012

(51) Int. Cl. A01H 5/00

(52) U.S. Cl. PLT/440

(57) ABSTRACT

A new and distinct Heuchera plant characterized by leaves that are red brown with hot pink spots in spring and when new to almost black with pink spots in older leaves summer through winter medium large, glossy leaves, well branched, medium large, mounding habit, and excellent vigor.

### BOTANICAL DENOMINATION

Heuchera hybrid

### VARIETY DESIGNATION

‘Galaxy’

### BACKGROUND OF THE INVENTION

The present invention relates to a new and distinct cultivar of Heuchera and given the cultivar name of ‘Galaxy’. Heuchera is in the family Saxifragaceae. Heuchera ‘Galaxy’ originated from a cross between Heuchera K428-5, a proprietary non-commercial hybrid, as the seed parent, and Heuchera K428-11, a proprietary non-commercial hybrid, as, as the pollen parent.

Compared to the seed parent, Heuchera K428-5, the new cultivar has glossy near black rather than dull brown leaves.

Compared to the pollen parent, Heuchera K428-11, the new cultivar has red black rather than red brown leaves.

Compared to Heuchera ‘Midnight Rose’, U.S. Plant Pat. No. 18,551, the new cultivar has leaves that are red brown with pink spots rather than black with pink spots, a larger habit, larger leaves, and fewer and shorter inflorescences.

This new Heuchera is uniquely distinguished by:

1. leaves that are red brown with hot pink spots in spring and when new to almost black with pink spots in older leaves summer through winter,

2. medium large, glossy leaves,

3. well branched, medium large, mounding habit,

4. excellent vigor.

This new cultivar has been reproduced only by asexual propagation (tip cuttings and tissue culture). Each of the progeny exhibits identical characteristics to the original plant. Asexual propagation by cuttings and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may change with variations in environment without a change in the genotype of the plant.

### BRIEF DESCRIPTION OF THE DRAWING

FIG. 1 shows a one-and-a-half-year-old plant of Heuchera ‘Galaxy’ growing in a two gallon container, grown in a cool greenhouse, in May in Canby, Oreg.

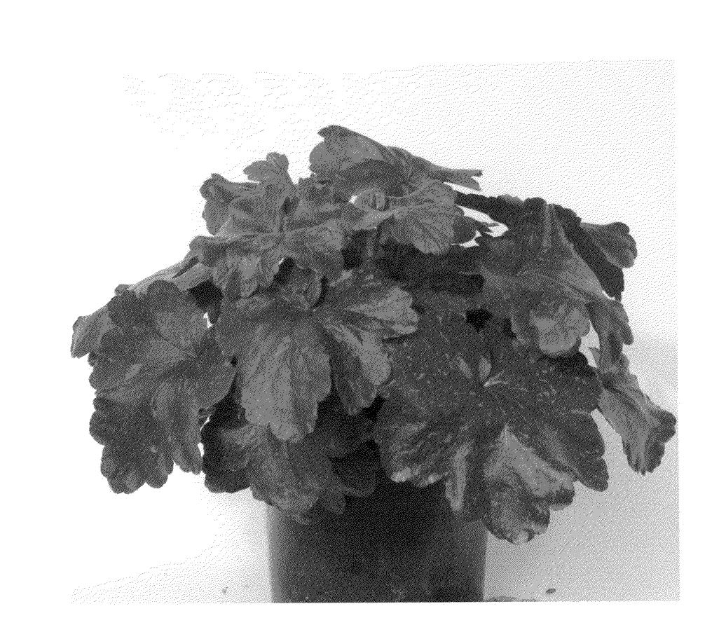

### DETAILED PLANT DESCRIPTION

The following is a detailed description of the new Heuchera cultivar based on observations taken in April of one-and-a-half-year-old specimens growing in one gallon containers, grown in a cool greenhouse, in late May in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, 5^{th} edition.
Plant:
   Form.—basal rosette, herbaceous perennial.
   Hardiness.—USDA Zone 4 to 9.
   Size.—27 cm tall from top of the foliage to the ground and 52 cm wide.
   Habit.—mounding.
   Vigor.—excellent.
Leaf:
   Type.—simple.
   Arrangement.—rosette.
   Shape.—ovate to orbicular.
   Lobing/division.—7 shallow lobes, shallow secondary lobes.
   Venation.—palmate.
   Margins.—broadly crenate.
   Apex.—mucronulate.
   Base.—cordate, lobes sometime overlapping at the base.
   Blade size.—grows to 17 cm long and 16 cm wide.
   Texture.—leathery.
   Surface texture.—glandular pubescent on both sides.
   Petiole description.—grows to 19 cm long and 3 mm wide, glandular hairs, Greyed Purple 187B.
   Leaf color.—topside, new leaves are Greyed Purple 187B with spots Red Purple 68A, bottom side Red Purple 59A; mature leaves topside are closest to Greyed Purple N186A with spots Red Purple 68C, bottom side Purple N79A.
Inflorescence:
   Size.—grows to 4 cm wide and 7 cm long.
   Type.—thyrse.
   Number of flowers per thyrse.—about 40.
   Number of thyrse.—about 3 in the first spring flush.
   Peduncle.—grows to 40 cm long and 3.5 mm wide at base, Greyed Purple 187A, glandular hairs.
   Pedicel.—2 mm long, with glandular hairs, Greyed Purple 187A.
   Bloom period.—May in Canby, Oreg.
Flower bud:
   Size.—2.5 mm wide and 4 mm long.
   Description.—glandular puberulent, ovoid, down facing.
   Color.—Greyed Red 185A.
Flower:
   Type.—perfect.
   Shape.—campanulate.
   Size.—7 mm long and 6 mm wide.
   Corolla description.—5 petals, 3 mm long and 1 mm wide, spatulate, reflexed, margins entire, tip acute, glabrous on top and glandular on bottom side, White N155B.
   Calyx description.—campanulate, 6 mm long and 4 mm wide, with 5 lobes divided ½ way to the base, each 1.5 mm wide and 2.5 mm long, glandular hairs outside and some glabrous inside, tip obtuse, margin entire; color both sides Green Yellow 1C, outside tinted with Greyed Red 181 A, but most strongly on the bottom half.
   Stamen description.—5 in number, filaments 4 mm long, White N155B, anthers 0.5 mm, Orange 26A, pollen Orange 31A.
   Pistil description.—1, 2-beaked, 4 mm long and 1.5 mm wide, Green Yellow 1A overall, ovary 1.5 mm long and 1.5 mm wide, style 2.5 mm long.
   Fragrance.—none.
   Lastingness.—each thyrse blooms for about 3 weeks on the plant.
Fruit:
   Type.—two-beaked ovoid capsule.
   Fertility.—low.
   Color.—Greyed Brown 199C.
Seed:
   Shape.—linear.
   Size.—2 mm long.
   Color.—Black 202A.
Pest and disease tolerance: The new cultivar is typical to the genus. No known resistances to pests or diseases. No problems have been found in Canby, Oreg.

I claim:

1. A new and distinct Heuchera plant as herein illustrated and described.
